SEQUENCE LISTING (10 sequences)

SEQ ID NO: 1 - RNA, 21 nt, synthetic construct
  source 1..21: mol_type = other RNA
gtgttcacaacgaaaccaatt

SEQ ID NO: 2 - RNA, 21 nt, synthetic construct
  source 1..21: mol_type = other RNA
ttggtttcgttgtgaacactt

SEQ ID NO: 3 - RNA, 21 nt, synthetic construct
  source 1..21: mol_type = other RNA
ctgattgataaggccatgatt

SEQ ID NO: 4 - RNA, 21 nt, synthetic construct
  source 1..21: mol_type = other RNA
tcatggccttatcaatcagtt

SEQ ID NO: 5 - RNA, 21 nt, synthetic construct
  source 1..21: mol_type = other RNA
gctgcagaggatgattgcttt

SEQ ID NO: 6 - RNA, 21 nt, synthetic construct
  source 1..21: mol_type = other RNA
agcaatcatcctctgcagctt

SEQ ID NO: 7 - DNA, 21 bp, synthetic construct
  source 1..21: mol_type = other DNA
ccaactcttttgtgccagaga

SEQ ID NO: 8 - DNA, 23 bp, synthetic construct
  source 1..23: mol_type = other DNA
ggctacattggtgttgagctttt

SEQ ID NO: 9 - DNA, 20 bp, synthetic construct
  source 1..20: mol_type = other DNA
cagggaggcagtgactcttc

SEQ ID NO: 10 - DNA, 20 bp, synthetic construct
  source 1..20: mol_type = other DNA
agtgtggaaagtgtggcgtt

(19) United States
(12) Patent Application Publication
(10) Pub. No.: US 2024/0207211 A1
(43) Pub. Date: Jun. 27, 2024

(54) COMPOSITION FOR PROMOTING BONE FORMATION OR TREATING BONE DISEASE BY ACTING ON OSTEOBLAST MITOCHONDRIA

(71) Applicant: SEOUL NATIONAL UNIVERSITY R&DB FOUNDATION, Seoul (KR)

(72) Inventors: Yun-Sil LEE, Seoul (KR); Joonho SUH, Seoul (KR)

(73) Assignee: SEOUL NATIONAL UNIVERSITY R&DB FOUNDATION, Seoul (KR)

(21) Appl. No.: 18/557,991

(22) PCT Filed: Apr. 29, 2022
(86) PCT No.: PCT/KR2022/006201

§ 371 Date: Oct. 30, 2023

(30) Foreign Application Priority Data
Apr. 30, 2021 (KR) 10-2021-0056626

(51) Int. Cl. A61K 31/194; A61K 45/06; A61P 19/08

(52) U.S. Cl. CPC A61K 31/194; A61K 45/06; A61P 19/08

(57) ABSTRACT

The present specification relates to an osteogenesis-promoting agent and/or a therapeutic agent for bone diseases comprising a pharmaceutical and/or food composition for promoting bone formation and/or preventing, treating, and/or ameliorating bone diseases, comprising an agent promoting the activation of osteoblasts as an active ingredient. Specifically, it was revealed for the first time that treatment with a low concentration of bongkrekic acid and/or regulation of the activity of Opa1 (suppression of expression), Fis1 (increase of expression), and Bax (increase of expression) genes induce morphological changes and fission of mitochondria to promote bone regeneration and/or bone formation, and the active ingredients can be utilized as a pharmaceutical composition for promoting bone regeneration and/or bone formation and a pharmaceutical composition for preventing and/or treating bone diseases.

### TECHNICAL FIELD

The present specification relates to a pharmaceutical composition comprising an agent promoting the activation of osteoblasts as an active ingredient for promoting bone formation and/or preventing, or treating bone diseases, and/or a food composition for promoting bone formation and/or preventing or ameliorating bone diseases.

### BACKGROUND

A large percentage of the elderly population that is rapidly increasing suffers from skeletal diseases involving osteoporosis or osteopenia. Numerous drugs have been developed to treat osteoporosis, suffered by approximately 1 million domestic patients, and the size of the therapeutic agent market is estimated to be about 200 billion Korean won in the domestic market and more than about 10 trillion Korean won in the global market.

Bone resorption inhibitors based on bisphosphonate, a representative osteoporosis therapeutic agent, are effective in increasing bone density, but serious side effects such as bisphosphonate-related osteonecrosis of the jaw (BRONJ) can occur. Therefore, research was conducted on the treatment of bone diseases using drugs that promote bone formation rather than inhibit bone resorption.

Mitochondria are cell organelles essential for the survival of all eukaryotic cells and are involved in the synthesis and regulation of adenosine triphosphate (ATP) as an energy source. A variety of functions within cells are regulated by mitochondria, including the control of cell signal processing, cell cycle, cell differentiation, cell growth, and apoptosis. Mitochondria constantly communicate with surrounding cell organelles and actively change their shape, and during this process, they undergo fission and change into small mitochondria, or generate mitochondrial-derived vesicles (MDVs).

Properly maintaining the form and function of the bones that make up the vertebrate skeleton is essential for maintaining life, and osteoblasts, which are bone-forming cells, secrete various substances to optimally maintain the condition of these bones. The present inventors discovered that activated osteoblasts secrete mitochondria or small MDVs into the surrounding area where bone is to be formed, and have filed an application for a related patent (application number: PCT/KR2020/007015) in 2020 for a pharmaceutical composition containing osteoblast-derived mitochondria as an active ingredient for promoting bone formation and treating bone diseases. The present inventors discovered that when osteoblasts are activated, mitochondria form a round donut shape, which increases MDV formation and secretion to promote bone formation.

Bongkrekic acid is a toxin produced by bacteria called Burkholderia gladioli found in fermented coconuts or corn, and inhibits cell function by preventing ATP from exporting out of the mitochondria, preventing cells from using ATP. Bongkrekic acid can induce death at high concentrations, and deaths due to this toxin have been reported in Indonesia and China. However, the present inventors confirmed for the first time that when osteoblasts are treated with low concentrations of bongkrekic acid, osteoblasts are activated and bone formation increases, confirming the possibility that bongkrekic acid, known as a poison, can also be used for medical purposes.

### SUMMARY OF THE INVENTION

### Technical Problem

The present inventors screened drugs that change the shape of mitochondria to a donut shape or increase mitochondrial secretion, and confirmed for the first time that bone formation can be induced by regulating the activities of bongkrekic acid, Opa1 and Fis1, which are important genes in regulating the shape of mitochondria, and Bax, a gene that acts on the mitochondrial outer membrane.

The present specification provides a composition for promoting bone formation and/or preventing, treating, and/or ameliorating bone diseases, containing an agent promoting activation of osteoblasts as an active ingredient, wherein the agent promoting activation of osteoblasts includes at least one selected from the group consisting of: 1) bongkrekic acid or a pharmaceutically acceptable salt thereof, 2) an inhibitor of Opa1 or a gene encoding the same, 3) an activator of Fis1 or a gene encoding the same, and 4) an activator of Bax or a gene encoding the same.

### Technical Solution

The present specification provides a composition for promoting bone formation and/or preventing, treating, and/or ameliorating bone diseases, containing an agent promoting the activation of osteoblasts as an active ingredient.

The composition may have an effect of promoting bone formation and/or preventing, treating and/or ameliorating bone diseases.

The present specification provides a method for promoting bone formation and/or preventing, treating and/or ameliorating bone diseases, including a step of administering an effective amount (e.g., a pharmaceutically effective amount) of an agent promoting the activation of osteoblasts and/or a composition containing the agent as an active ingredient for promoting bone formation and/or preventing, treating and/or ameliorating bone diseases, to a subject in need of bone formation or promoting bone formation, and/or preventing, treating and/or ameliorating bone diseases.

The present specification provides a use of an agent promoting the activation of osteoblasts and/or a composition containing the agent as an active ingredient for promoting bone formation and/or preventing, treating and/or ameliorating bone diseases, for bone formation and/or preventing, treating and/or ameliorating bone diseases.

The present specification provides a use of an agent promoting the activation of osteoblasts and/or a composition containing the agent as an active ingredient for promoting bone formation and/or preventing, treating and/or ameliorating bone diseases in the manufacture of a composition (e.g., a food composition and/or pharmaceutical composition) for bone formation and/or preventing, treating and/or ameliorating bone diseases.

In the present specification, “bone disease” may include, but is not limited to, fractures, osteoclasia, osteoporosis, osteomalacia, osteoarthritis, rheumatoid arthritis, and/or osteogenesis imperfecta.

In the present specification, Opa1, Fis1 (Mitochondrial fission 1), and Bax (bcl-2-associated X) may refer to proteins encoded by each gene, but are not limited thereto, and in particular, the protein encoded by the Opa1 gene may be a dynamin-like 120 kDa protein.

In the present specification, Alp1 may refer to both the Alp1 gene and the enzyme (Alkaline phosphatase, tissue-nonspecific isozyme) encoded by the gene, and the enzyme is directly related to bone diseases such as hypophosphatasia and hypercalcemia.

In the present specification, Ibsp may refer to both the Ibsp gene and the bone sialoprotein encoded by the gene, and regulation of the gene is important for mineralization of bone matrix and growth of bone tumors.

In the present specification, “bongkrekic acid” is a respiratory toxin produced from fermented coconut or corn contaminated by the bacteria Burkholderia gladioli pathovar cocovenenans, and is highly toxic because it inhibits the ADP/ATP translocase, also called the mitochondrial ADP/ATP carrier, preventing ATP from leaving the mitochondria and providing metabolic energy to the rest of the cell.

In the present specification, proteins may include forms such as antibodies, antibody fragments, or analogs thereof, but are not limited thereto.

In the present specification, genes may include forms such as DNA, RNA (e.g., siRNA, microRNA, shRNA, etc.), PNA (peptide nucleic acid), aptamer, etc., but are not limited thereto.

In the present specification, mRNA may be in an artificially synthesized form or a naturally obtainable form, but is not limited thereto.

### Pharmaceutical Use

One example provides a pharmaceutical composition containing an agent promoting activation of osteoblasts as an active ingredient, wherein the agent promoting activation of osteoblasts includes at least one selected from the group consisting of: 1) bongkrekic acid or a pharmaceutically acceptable salt thereof, 2) an inhibitor of Opa1 or a gene encoding the same, 3) an activator of Fis1 or a gene encoding the same, and 4) an activator of Bax or a gene encoding the same.

The inhibitor of Opa1 or a gene encoding the same of the pharmaceutical composition may be at least one selected from the group consisting of: small-interfering RNAs (siRNAs), microRNAs (miRNAs), antisense oligonucleotides (ASOs), and aptamers targeting the Opa1 gene, but is not limited thereto.

The activator of Fis1 or a gene encoding the same of the pharmaceutical composition may be at least one selected from the group consisting of mRNA encoding the Fis1 gene and a recombinant vector carrying the same, but is not limited thereto.

The activator of Bax or a gene encoding the same of the pharmaceutical composition may be at least one selected from the group consisting of mRNA encoding the Bax gene and a recombinant vector carrying the same, but is not limited thereto.

The pharmaceutical composition may increase the proportion of osteoblast mitochondria that have changed to a donut shape, but is not limited thereto.

The “donut shape” may mean, among osteoblast mitochondria, one having a form in which the circularity value ((4×area of mitochondria)/(π×(FeretMax)²)) is 0.5 to 1 and/or one or more pores are formed (FeretMax means maximum ferret diameter).

The pharmaceutical composition may increase the proportion of osteoblast mitochondria in which the circularity value is 0.5 to 1 and/or one or more pores are formed, but is not limited thereto.

The pharmaceutical composition may include the bongkrekic acid at a concentration of 0.1 to 1000 μM, 0.1 to 500 μM, 0.1 to 200 μM, 0.1 to 100 μM, 0.1 to 70 μM, 0.1 to 50 μM, 0.1 to 45 μM, 1 to 1000 μM, 1 to 500 μM, 1 to 200 μM, 1 to 100 μM, 1 to 70 μM, 1 to 50 μM, 1 to 45 μM, 10 to 1000 μM, 10 to 500 μM, 10 to 200 μM, 10 to 100 μM, 10 to 70 μM, 10 to 50 μM, 10 to 45 μM, 30 to 1000 μM, 30 to 500 μM, 30 to 200 μM, 30 to 100 μM, 30 to 70 μM, 30 to 50 μM, 30 to 45 μM, 35 to 1000 μM, 35 to 500 μM, 35 to 200 μM, 35 to 100 μM, 35 to 70 μM, 35 to 50 μM or 35 to 45 μM, for example, 40 μM, but is not limited thereto.

The pharmaceutical composition may have an effect of increasing the expression of at least one selected from the group consisting of Alp1 and Ibsp, but is not limited thereto.

The pharmaceutical composition may be a pharmaceutical composition for promoting bone formation and/or preventing and/or treating bone diseases, but is not limited thereto.

In this specification, “treatment” is used to include amelioration, alleviation or stabilization of disease state or symptoms, partial or complete recovery, prolongation of survival, reduction of disease extent, delay or alleviation of disease progression, and other beneficial treatment results. “Prevention” is used to include all mechanisms and/or effects that act on subjects who do not have a specific disease but act to prevent the specific disease from developing, delay its onset, or reduce the frequency of its occurrence.

The content of the agent promoting the activation of osteoblasts used as an active ingredient in the pharmaceutical composition provided in this specification can be appropriately adjusted depending on the form and purpose of use, the condition of the subject of use, and the type and severity of symptoms, and may be 0.001 to 99.9% by weight, 0.001 to 90% by weight, 0.001 to 75% by weight, 0.001 to 50% by weight, 0.01 to 99.9% by weight, 0.01 to 90% by weight, 0.01 to 75% by weight, 0.01 to 50% by weight, 0.1 to 99.9% by weight, 0.1 to 90% by weight, 0.1 to 75% by weight, 0.1 to 50% by weight, 1 to 99.9% by weight, 1 to 90% by weight, 1 to 75% by weight, 1 to 50% by weight, 5 to 99.9% by weight %, 5 to 90% by weight, 5 to 75% by weight, 5 to 50% by weight, 10 to 99.9% by weight, 10 to 90% by weight, 10 to 75% by weight, or 10 to 50% by weight, based on the weight of solid content (solid content obtained by removing the solvent from the extract), but is not limited thereto.

In addition, the content of the agent promoting the activation of osteoblasts may be 0.001 to 400 mg/kg (body weight; BW), 1 to 400 mg/kg, 50 to 400 mg/kg, 100 to 400 mg/kg, 0.001 to 300 mg/kg, 1 to 300 mg/kg, 50 to 300 mg/kg, 100 to 300 mg/kg, 0.001 to 200 mg/kg, 1 to 200 mg/kg, 50 to 200 mg/kg, 100 to 200 mg/kg, 0.001 to 100 mg/kg, 1 to 100 mg/kg or 50 to 100 mg/kg, based on the body weight of the subject administering the pharmaceutical composition, but is not limited thereto.

The pharmaceutical composition or agent promoting the activation of osteoblasts, which is the active ingredient, may be administered by various routes to a subject selected from vertebrates such as primates such as humans and monkeys; rodents such as mice, rats, and rabbits; mammals including dogs, cats, cattle, pigs, sheep, horses, and goats; birds including chickens, ducks and geese; reptiles including snakes, lizards, turtles, and alligators; amphibians; and fish, but is not limited thereto.

The method of administration of the pharmaceutical composition may be any commonly used method, for example, parenteral administration such as intravenous, intramuscular, subcutaneous injection, or local injection. In one example, the pharmaceutical composition may be administered (e.g., in the form of an injection, etc.) to an area requiring bone formation or a bone disease area, but is not limited thereto.

The pharmaceutical composition may be used by formulating as an oral dosage form such as powders, granules, tablets, capsules, suspensions, emulsions, syrups, and aerosols or by formulating as a parenteral formulation in the form of an injection (e.g., sterile injection solution, etc.), or the like according to conventional methods, but is not limited thereto.

In addition to the above-described active ingredients, the pharmaceutical composition may be administered in a mixture with one or more supplements selected from the group consisting of pharmaceutically and/or physiologically acceptable carriers, excipients, diluents, and the like, generally selected considering the administration method and standard pharmaceutical practice. For example, the type of the pharmaceutically and/or physiologically acceptable carrier is not particularly limited, and any carrier commonly used in the art may be used, and it may include one or more types selected from the group consisting of saline solution, sterile water, Ringer's solution, buffered saline solution, albumin injection solution, glycerol, ethanol lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, mineral oil, and the like, but is not limited thereto, and may be any carrier commonly used in the pharmaceutical field.

The pharmaceutically and/or physiologically acceptable diluent and/or excipient may be one or more selected from the group consisting of all fillers, extenders, binding agents, wetting agents, disintegrating agents, lubricants, surfactants, etc. commonly used in the proper formulation of pharmaceutical compositions, but are not limited thereto.

For example, in the case of solid agents for oral administration such as tablets, pills, powders, granules, or capsules, the excipient may be at least one material for the formulation of such solid agents, such as one or more selected from the group consisting of starch, calcium carbonate, sucrose, lactose, gelatin, etc. In addition to these, lubricants such as magnesium stearate talc and the like may also be used. In addition, in the case of formulation of liquid agents for oral administration such as suspending agents, oral solutions, emulsions, and syrups, one or more types selected from the group consisting of commonly used simple diluents such as water and liquid paraffin may be used, and in addition to these, various commonly used excipients, such as wetting agents, sweetening agents, flavoring agents, and/or preservatives, may be included, but are not limited thereto.

For example, the pharmaceutical composition may be administered orally, intraorally, or sublingually in the form of a tablet containing starch or lactose, or in the form of a capsule containing appropriate excipients, or in the form of an elixir or suspending agent containing flavoring or coloring chemicals. These liquid agents may be formulated with pharmaceutically acceptable additives such as suspending agents (for example, mixtures of methylcellulose, semisynthetic glycerides such as Wittepsol or Apricot kernel oil and PEG-6 esters, or glyceride mixtures, such as a mixture of PEG-8 and caprylic/capric glycerides), but are not limited thereto.

Parenteral agents using the pharmaceutical composition of the present specification may be injection solutions, suppositories, powders for respiratory inhalation, aerosols for sprays, ointments, powders for application, oils, creams, etc., but are not limited thereto.

To formulate the pharmaceutical composition of the present specification for parenteral administration, sterilized aqueous solutions, non-aqueous solvents, suspending agents, emulsions, freeze-dried agents, external applications, etc. may be used, and for the non-aqueous solvents and suspending agents, propylene glycol, polyethylene glycol, vegetable oils such as olive oil, and injectable esters such as ethyl oleate may be used, but they are not limited thereto.

When the pharmaceutical composition of the present specification is formulated as an injection, the pharmaceutical composition of the present specification may be mixed in water with a stabilizer or buffer to prepare a solution or suspension, which may be formulated for unit administration in ampoules or vials, but it is not limited thereto.

When the pharmaceutical composition of the present specification is formulated as an aerosol, propellants, etc. may be mixed with additives to disperse water-dispersed concentrates or wet powders.

When the pharmaceutical composition of the present specification is formulated into ointment, cream, powder for application, oil, external skin application, etc., it may be formulated using animal oil, vegetable oil, wax, paraffin, starch, tracant, cellulose derivatives, polyethylene glycol, silicone, bentonite, silica, talc, zinc oxide, etc. as carriers, but it is not limited thereto.

The pharmaceutically effective amount and effective dosage of the pharmaceutical composition of the present specification may vary depending on the formulation method, administration method, administration time, and/or administration route of the pharmaceutical composition, and may vary depending on various factors including the type and degree of reaction to be achieved by administration of the pharmaceutical composition, the type of subject to be administered, age, weight, general health status, symptoms or degree of disease, gender, diet, excretion, drugs and other composition components used simultaneously or sequentially in the subject, etc., and similar factors well known in the pharmaceutical field, and a person skilled in the art may easily determine and prescribe an effective dosage for the desired treatment, but they are not limited thereto.

The pharmaceutical composition of the present specification may be administered once a day, or may be administered in several divided doses. The pharmaceutical composition of the present specification may be administered as an individual therapeutic agent or in combination with other therapeutic agents, and may be administered sequentially or simultaneously with conventional therapeutic agents. In consideration of all of the above factors, it may be administered in an amount that can achieve maximum effect with the minimum amount without side effects, and this can be easily determined by a person skilled in the art.

The administration route and administration method of the pharmaceutical composition of the present specification may be independent, and as long as the pharmaceutical composition can reach the desired area, any administration route and method may be used without particular limitation. The pharmaceutical composition may be administered by oral administration or parenteral administration, but is not limited thereto.

As a parenteral administration method of the pharmaceutical composition of the present specification, intravenous administration, intraperitoneal administration, intramuscular administration, transdermal administration, or subcutaneous administration may be used, and a method of applying the composition to a disease area or spraying or inhaling the composition may also be used, but is not limited thereto.

The pharmaceutical composition of the present specification may be used alone, but in order to increase the therapeutic efficiency, it may be additionally used in combination with various skin aging prevention or treatment methods and/or wound treatment methods such as radiation therapy, chemotherapy, etc., but is not limited thereto.

### Food Composition

One example provides a food composition containing an agent promoting activation of osteoblasts as an active ingredient, wherein the agent promoting activation of osteoblasts includes at least one selected from the group consisting of: 1) bongkrekic acid or a pharmaceutically acceptable salt thereof, 2) an inhibitor of Opa1 or a gene encoding the same, 3) an activator of Fis1 or a gene encoding the same, and 4) an activator of Bax or a gene encoding the same.

The inhibitor of Opa1 or a gene encoding the same of the food composition may be at least one selected from the group consisting of: small-interfering RNAs (siRNAs), microRNAs (miRNAs), antisense oligonucleotides (ASOs), and aptamers targeting the Opa1 gene, but is not limited thereto.

The activator of Fis1 or a gene encoding the same of the food composition may be at least one selected from the group consisting of mRNA encoding the Fis1 gene and a recombinant vector carrying the same, but is not limited thereto.

The activator of Bax or a gene encoding the same of the food composition may be at least one selected from the group consisting of mRNA encoding the Bax gene and a recombinant vector carrying the same, but is not limited thereto.

The food composition may increase the proportion of osteoblast mitochondria that have changed to a donut shape, but is not limited thereto.

The “donut shape” may mean, among osteoblast mitochondria, one having a form in which the circularity value ((4×area of mitochondria)/(π×(FeretMax)²)) is 0.5 to 1 and/or one or more pores are formed (FeretMax means maximum ferret diameter).

The food composition may increase the proportion of osteoblast mitochondria in which the circularity value is 0.5 to 1 and/or one or more pores are formed, but is not limited thereto.

The food composition may include the bongkrekic acid at a concentration of 0.1 to 1000 μM, 0.1 to 500 μM, 0.1 to 200 μM, 0.1 to 100 μM, 0.1 to 70 μM, 0.1 to 50 μM, 0.1 to 45 μM, 1 to 1000 μM, 1 to 500 μM, 1 to 200 μM, 1 to 100 μM, 1 to 70 μM, 1 to 50 μM, 1 to 45 μM, 10 to 1000 μM, 10 to 500 μM, 10 to 200 μM, 10 to 100 μM, 10 to 70 μM, 10 to 50 μM, 10 to 45 μM, 30 to 1000 μM, 30 to 500 μM, 30 to 200 μM, 30 to 100 μM, 30 to 70 μM, 30 to 50 μM, 30 to 45 μM, 35 to 1000 μM, 35 to 500 μM, 35 to 200 μM, 35 to 100 μM, 35 to 70 μM, 35 to 50 μM or 35 to 45 μM, for example, 40 μM, but is not limited thereto.

The food composition may have an effect of increasing the expression of at least one selected from the group consisting of Alp1 and Ibsp, but is not limited thereto.

The food composition may be a food composition for promoting bone formation and/or preventing and/or ameliorating bone diseases, but is not limited thereto.

In the food compositions provided in the present specification, the term “food” refers to an edible natural product or processed product containing one or more nutrients, and in a general sense, may be used to mean one or more selected from the group consisting of various general foods, health functional foods, beverages, food additives, beverage additives, etc. The term “food composition” may refer to a combination of ingredients for producing the food. In one example, the food may refer to a health functional food.

The content of the agent promoting the activation of osteoblasts used as an active ingredient in the food composition may be appropriately adjusted depending on the type and purpose of food use, etc., and may be 0.00001 to 99.9% by weight, 0.0001 to 99.9% by weight, 0.001 to 99.9% by weight, or 0.001 to 50% by weight, based on the weight of solid content, but is not limited thereto.

In addition, the content of the agent promoting the activation of osteoblasts may be 0.001 to 400 mg/kg (body weight; BW), 1 to 400 mg/kg, 50 to 400 mg/kg, 100 to 400 mg/kg, 0.001 to 300 mg/kg, 1 to 300 mg/kg, 50 to 300 mg/kg, 100 to 300 mg/kg, 0.001 to 200 mg/kg, 1 to 200 mg/kg, 50 to 200 mg/kg, 100 to 200 mg/kg, 0.001 to 100 mg/kg, 1 to 100 mg/kg or 50 to 100 mg/kg, based on the body weight of the subject administering the food composition, but is not limited thereto.

The “subject” of administration of the food composition may be an individual selected from vertebrates such as primates such as humans and monkeys; rodents such as mice, rats, and rabbits; mammals including dogs, cats, cattle, pigs, sheep, horses, and goats; birds including chickens, ducks and geese; reptiles including snakes, lizards, turtles, and alligators; amphibians; and fish; or a cell, tissue, cell culture, or the like isolated from the individual.

### Advantageous Effects

The composition of the present specification can promote the activation of osteoblasts, and specifically promote bone formation and/or prevent, treat, and/or ameliorate bone diseases.

### BRIEF DESCRIPTION OF THE DRAWINGS

FIGS. 1A to 1C are photographs showing the results of observing osteoblast mitochondria after transfecting siOpa1, pCMV-fis1, and siFis1 into osteoblasts, respectively or adding bongkrekic acid. siCtrl refers to the control for siOpa1 and siFis1, Ctrl (Tris) refers to the control for bongkrekic acid, and pCMV-Ctrl refers to the control for pCMV-fis1.

FIGS. 1D and 1E are graphs showing the results of quantitative analysis of morphological changes in osteoblast mitochondria after transfection of siOpa1, pCMV-fis1, and siFis1 into osteoblasts, respectively, or addition of bongkrekic acid. siCtrl refers to the control for siOpa1 and siFis1, Ctrl refers to the control for bongkrekic acid, and pCMV-Ctrl refers to the control for pCMV-fis1.

FIGS. 2A, 3B, 3C, and 4A are photographs showing the results of observing osteoblasts stained with alkaline phosphatase (ALP) after transfecting siOpa1, siFis1, and pCMV-Fis1 into osteoblasts, respectively or adding bongkrekic acid.

FIGS. 2B, 3B, 3D, and 4B are graphs showing the results of confirming the expression of osteogenic markers by transfecting siOpa1, siFis1, and pCMV-Fis1 into osteoblasts, respectively or adding bongkrekic acid, and then performing qRT-PCR.

FIGS. 5A and 5B are photographs showing the results of observing osteoblasts stained with alkaline phosphatase (ALP) after transfection of siBax and pCMV-Bax into osteoblasts, respectively.

FIG. 6 is a mimetic diagram expressing the effects of Opa1, Fis1, Bax, and bongkrekic acid on mitochondria and resulting osteogenesis.

### DETAILED DESCRIPTION OF THE EMBODIMENTS

Hereinafter, the present invention is described in detail with reference to the following examples. However, the following examples are only intended to illustrate the present invention, and the present invention is not limited by the following examples.

All values of the statistical analysis performed in the following examples were expressed as mean±standard error of the mean (SEM), one-way analysis of variance with Tukey's post hoc test was performed for statistical analysis of two or more groups, and Student's t tests were performed for two groups of statistical analysis. If P<0.05, it was considered significant.

### Example 1. Extraction of Skull-Derived Osteoblasts

To confirm changes in the morphology of osteoblast mitochondria, transgenic mice were prepared to express green fluorescent protein (GFP) specifically in the mitochondrial matrix. Specifically, lgs1^{CKI-mitoGFP/CKI-mitoGFP} knock-in C57BL/6 female mice were provided by Jeremy Nathans (Johns Hopkins University School of Medicine, MD), Col1a1-Cre male mice were provided by the Korea Research Institute of Bioscience and Biotechnology, and Col1a1-Cre was prepared to be expressed in these mice using the 2.3 kb murine Col1a1 promoter. By mating the prepared C57BL/6 female transgenic mouse with the Col1a1-Cre male mouse, newborn transgenic mice (Col1a1-Cre; Igs1^{CKI-mito-GFP/+}) that conditionally express GFP only in the mitochondria of osteoblasts were prepared. All animal studies were approved by the Seoul National University Institutional Animal Care and Use Committees.

The skull cells of the prepared transgenic mice that express GFP conditionally only in the mitochondria were treated with 0.25% trypsin (Gibco) for 10 minutes, incubated in 2 mg/mL type 2 collagenase (Worthington) for 30 minutes, and in fresh type 2 collagenase for another 60 minutes. The cells separated from the bone tissue were collected by centrifugation at 1300 rpm for 3 minutes, and the cells were placed on serum and centrifuged to remove debris. To separate living osteoblasts that express GFP in mitochondria, they were stained with 7AAD (Bio-Legend) for 10 minutes (only dead cells were stained) and a flow cytometry technique (Fluorescence-activated cell sorting, FACS) was used. Cells having similar shapes and sizes were separated, and only living cells (7-AAD negative cells) were selected from them, and after dividing them into osteoblasts that express GFP and cells that do not express GFP, only the osteoblasts that express GFP were collected.

### Example 2. Confirmation of Mitochondrial Morphological Changes During the Differentiation Process of Osteoblasts

### Examples 2-1. Osteoblast Differentiation and Transfection

The osteoblasts extracted in Example 1 were induced to be differentiated in a medium composed of alpha MEM (Minimum Essential Media, Hyclone) supplemented with 10% fetal bovine serum (Gibco) and 100 U/mL penicillin-streptomycin (Gibco) for 5 days. Specifically, the differentiation medium is composed of alpha MEM with 50 μg/mL ascorbic acid (Amreasco), 5 mM beta-glycerophosphate (Sigma), 10% fetal bovine serum (Gibco), and 100 U/mL penicillin-streptomycin (Gibco). Osteoblast differentiation was induced for 5 days.

Short interfering RNA (siRNA) that inhibits the expression of the Opa1 (dynamin-like 120 kDa protein) gene, which induces mitochondrial fusion, and the Fis1 (Mitochondrial fission 1 protein) gene, which induces mitochondrial fission (siOpa1, siFis1, respectively) was transfected into 300,000 osteoblasts per 1 mL at 30 nM each at the same time as differentiation induction using Lipofectamine RNAiMAX Reagent (Invitrogen), and to increase mitochondrial fission, the pCMV-Fis1 plasmid (Korea Human Gene Bank) was transfected into 300,000 osteoblasts per mL at 1 μg/mL at the same time as differentiation induction using TransIT®-LT1 Transfection Reagent (Mirus) to overexpress the Fis1 gene. In addition, the osteoblasts prepared above were treated simultaneously with differentiation with Tris solution-based bongkrekic acid (Sigma, B6179, USA) at a concentration of 40 μM. As a control for the siRNA, osteoblasts were prepared by transfecting the control siRNA in substantially the same manner as the siRNA; as a control for the plasmid, osteoblasts were prepared by transfecting the pCMV-SPORT6 empty vector plasmid into which no gene was inserted in substantially the same manner as the pCMV-Fis1 plasmid; and as a control for the bongkrekic acid, osteoblasts treated with 0.01M Tris solution were prepared. The siRNAs were purchased from Bioneer, Korea and transfected using Lipofectamine RNAimax (Invitrogen, USA) according to the manufacturer's instructions, and specific information on the siRNAs is shown in Table 1 below.

TABLE 1

| | ID number | RNA | Nucleotide | SEQ |
|---|---|---|---|---|
| siRNA name | (Bioneer siRNA IDs) | accession | sequence (5′ → 3′) | ID NO |
| siOpa1 | 74143-1 | NM_001199177.1 | guguucacaacgaaaccaatt | 1 |
| Forward | | NM_133752.3 | | |
| siOpa1 | | | uugguuucguugugaacactt | 2 |
| Reverse | | | | |
| siFis1 | 66437-1 | NM_001163243.1 | cugauugauaaggccaugatt | 3 |
| Forward | | NM_001347504.1 | | |
| siFis1 | | NM_025562.3 | ucauggccuuaucaaucagtt | 4 |
| Reverse | | | | |
| control | SN-1002 | — | — | — |
| siRNA | (catalog | | | |
| Forward | number) | | | |
| control | | | — | — |
| siRNA | | | | |
| Reverse | | | | |

The pCMV-Fis1 plasmid containing full-length mouse Fis1 cDNA (ID: mMU002771) was purchased from the Korea Human Gene Bank, and was transfected into osteoblasts using TransIT-LT1 transfection reagent (Mirus Bio, USA) according to the manufacturer's instructions. The pCMV-SPORT6 empty vector plasmid was made by removing the Fis1 gene from pCMV-Fis1 (ID: mMU002771) with EcoRI and MfeI restriction enzymes and reattaching it.

### Examples 2-2. Osteoblast Fluorescence Imaging

Osteoblasts prepared through transfection or drug treatment in Example 2-1 and each control were fixed in 4% paraformaldehyde solution and mounted with DAPI Fluoremount-G® (Southern Biotech, 0100-20, USA), and nuclei were stained. Images were taken in Airyscan mode using a Zeiss LSM 800 confocal microscope (Zeiss, Germany) equipped with a Plan-Apochromat 63×/1.4 numerical aperture oil objective lens. The photographed osteoblast sample was cultured in a glass bottom dish (MatTek; P35G-1.5-14-C, USA, or SPL; 101350, Korea). In all imaging, the 488 nm laser was used to detect mitochondrial matrix GFP, and the 405 nm laser was used to detect DAPI. The captured images were analyzed for mitochondrial length (using the fiber length function) and the number of donut-shaped mitochondria in osteoblasts using Zeiss Zen 3.1 (blue edition) software (Zeiss, Germany). Mitochondria were judged to be donut-shaped if the following two conditions were met: the circularity value was 0.5 to 1, and the number of holes was 1 or more. All analyses were manually analyzed by the above software.

### Examples 2-3. Result

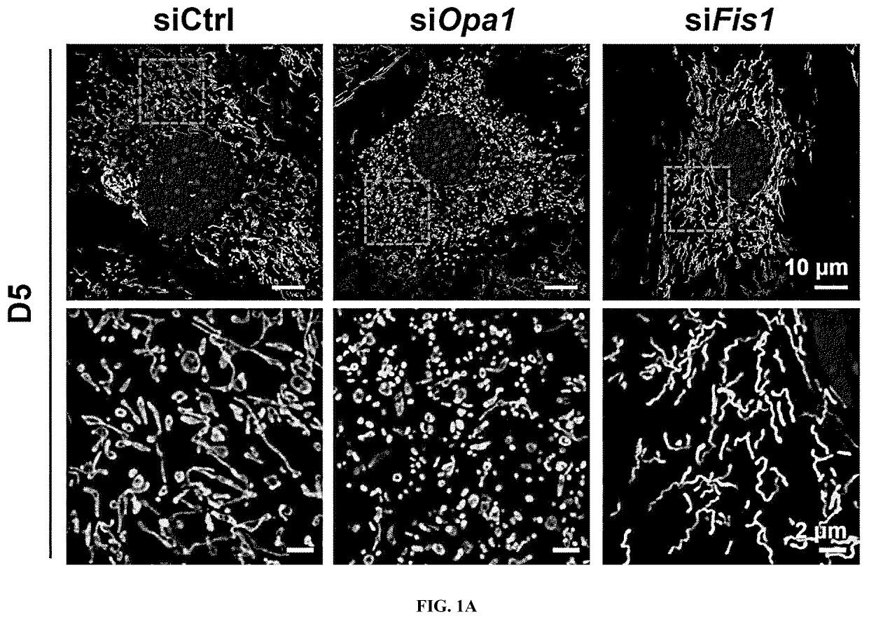

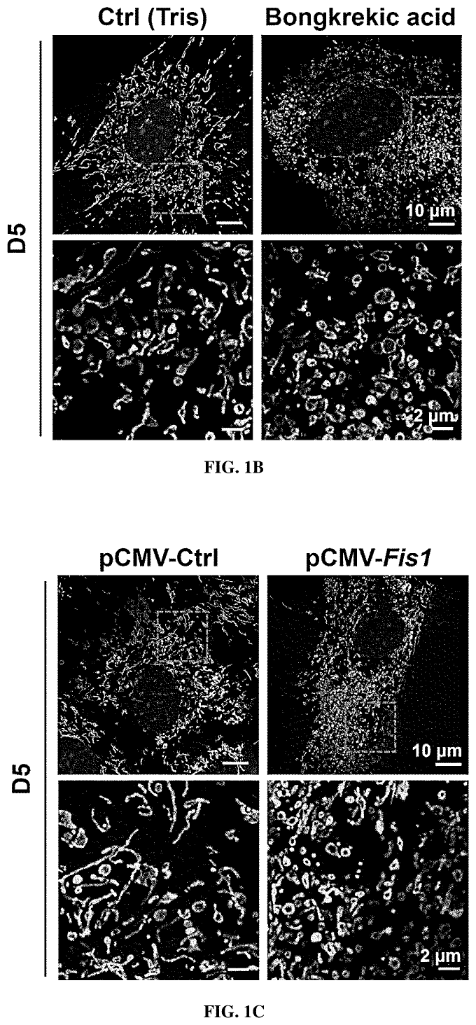

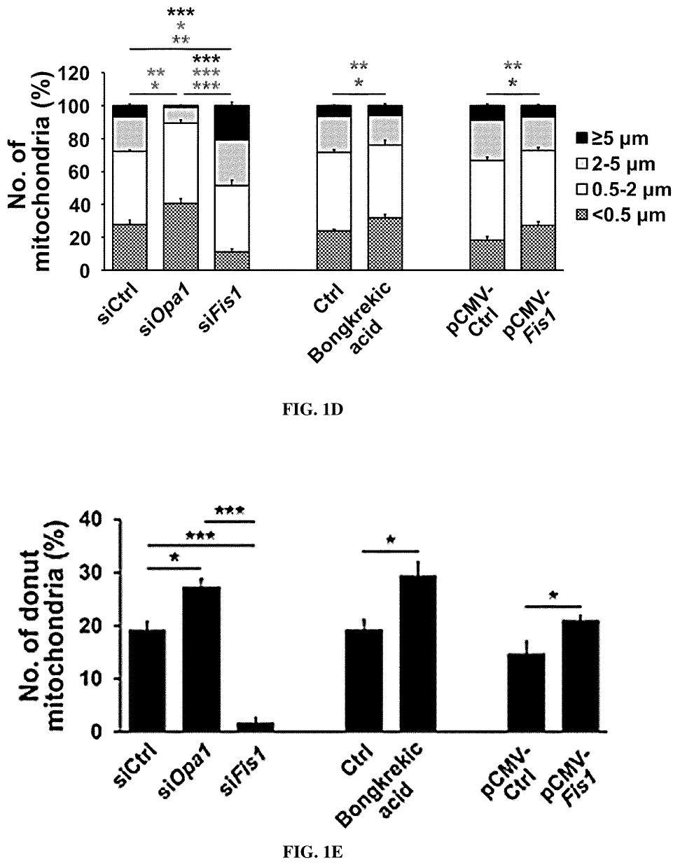

The results of images taken using a Zeiss LSM 800 confocal microscope (Zeiss, Germany) in Example 2-2 are shown in FIGS. 1a to 1c. In osteoblasts transfected with siOpa1 and pCMV-Fis1 plasmids, respectively, and osteoblasts added with bongkrekic acid, mitochondrial fission and formation of donut-shaped mitochondria increased compared to the control, and in osteoblasts transfected with siFis1, mitochondrial fission and formation of donut-shaped mitochondria were significantly reduced compared to the control.

The results of quantitative analysis of mitochondrial morphological changes in Example 2-2 using Zeiss Zen 3.1 software (Zeiss) are shown in FIGS. 1D and 1E. Mitochondrial fission was significantly increased in osteoblasts transfected with siOpa1 and pCMV-Fis1 plasmids, respectively, and in osteoblasts added with bongkrekic acid, compared to the respective controls, and in osteoblasts transfected with siFis1, mitochondrial fission was significantly reduced compared to the control. In addition, in osteoblasts transfected with siOpa1 and pCMV-Fis1 plasmids, respectively and osteoblasts added with bongkrekic acid, the number of donut-shaped mitochondria was significantly increased compared to the respective controls, and in osteoblasts transfected with siFis1, the number of donut-shaped mitochondria were significantly reduced compared to the control.

### Example 3. Evaluation of Osteogenic Activity of Osteoblasts

### Examples 3-1. Osteoblast Transfection and Differentiation

The osteoblasts extracted in Example 1 were transfected with 30 nM of siOpa1, siFis1, and 1 μg/mL of pCMV-Fis1 plasmids, respectively, in substantially the same manner as in Example 2-1, and separate osteoblasts were treated with 40 μM bongkrekic acid, and additionally, the osteoblasts extracted in Example 1 were transfected with 30 nM siBax, which is an siRNA of Bax (Bcl-2-associated X), and 1 μg/mL pCMV-Bax plasmid (Korea Human Gene Bank, ID: mMU001857), respectively, and differentiation of the osteoblasts was induced for 5 days in a medium consisting of alpha MEM (Minimum Essential Media, Hyclone) supplemented with 10% fetal bovine serum (Gibco) and 100 U/mL of penicillin-streptomycin (Gibco) of Example 2-1. As a control for the siRNA, osteoblasts were prepared by transfecting the control siRNA of Example 2-1 in substantially the same manner as the siRNA; as a control for the plasmid, osteoblasts were prepared by transfecting the pCMV-SPORT6 empty vector plasmid into which no gene was inserted in substantially the same manner as the pCMV-Fis1 plasmid; and as a control for the bongkrekic acid, osteoblasts treated with 0.01M Tris solution were prepared. Specific information on siBax, which is the siRNA of Bax (Bcl-2-associated X), is shown in Table 2 below.

TABLE 2

| | ID number | RNA | Nucleotide | SEQ |
|---|---|---|---|---|
| siRNA name | (Bioneer siRNA IDs) | accession | sequence (5′ → 3′) | ID NO |
| siBax | 12028-1 | NM_007527.3 | gcugcagaggaugauugcutt | 5 |
| Forward | | | | |
| siBax | | | agcaaucauccucugcagctt | 6 |
| Reverse | | | | |

### Examples 3-2. Alkaline Phosphatase (ALP) Staining

To confirm the osteogenic activity of the osteoblasts differentiation-induced in Example 3-1, staining of alkaline phosphatase (ALP), an osteogenic marker, was performed and observed. Specifically, 1 mg of Naphthol AS-MX phosphate (Sigma) was dissolved in 100 μL of N,N-dimethylformamide (Sigma), then 2 mL of 0.1% Fast blue BB salt (Sigma) solution was added and ALP staining was performed at 37° C. for 30 minutes. Osteogenic activity was confirmed by the intensity of staining, and it was evaluated that the darker the staining, the higher the expression of ALP.

### Examples 3-3. Real-Time Polymerase Chain Reaction (Quantitative RT-PCR, qRT-PCR)

qRT-PCR was performed to evaluate the gene expression level of the osteoblasts that underwent ALP staining in Example 3-2. Total RNA was extracted from the cells using QIAzol Lysis Reagent (Qiagen, Germany) and RNeasy Mini Kit (Qiagen, Germany), and cDNA was prepared using PrimeScript RT Reagent Kit (Takara, Japan) according to the manufacturer's instructions. Quantitative RT-PCR was performed using TB Green Premix Ex Taq II (Takara, Japan) and StepOnePlus Real-Time PCR system (Applied Biosystems, USA). Relative gene expression was determined using standard 2(−ΔΔCt) calculations with normalization to 18S rRNA, and information on the primers used is specifically shown in Table 3 below.

TABLE 3

| | Nucleotide | SEQ |
|---|---|---|
| Gene name | sequence (5′ → 3′) | ID NO |
| Alpl F_primer | ccaactcttttgtgccagaga | 7 |
| Alpl R_primer | ggctacattggtgttgagctttt | 8 |
| Ibsp F_primer | cagggaggcagtgactcttc | 9 |
| Ibsp R_primer | agtgtggaaagtgtggcgtt | 10 |

### Examples 3-4. Evaluation of Osteoblast Staining Degree and Differentiation-Related Gene Expression Levels

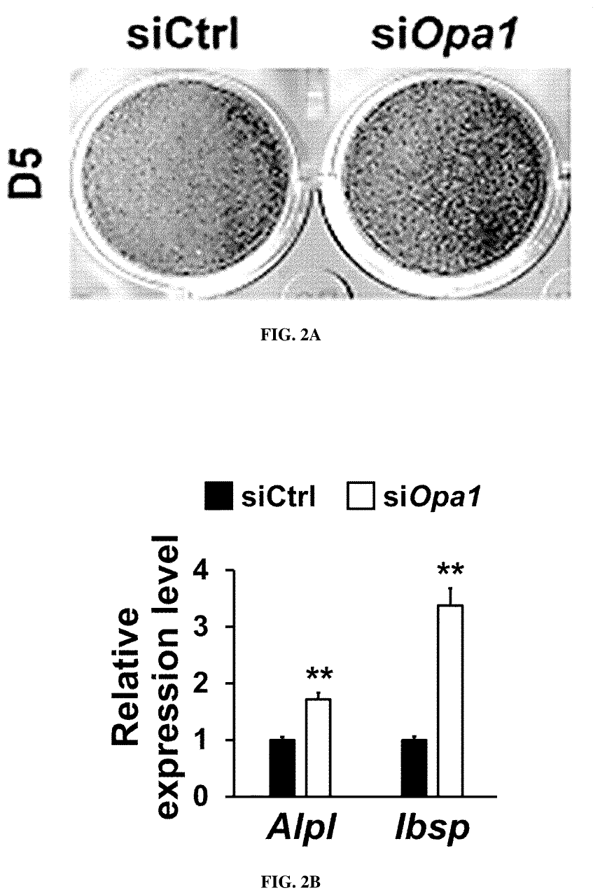

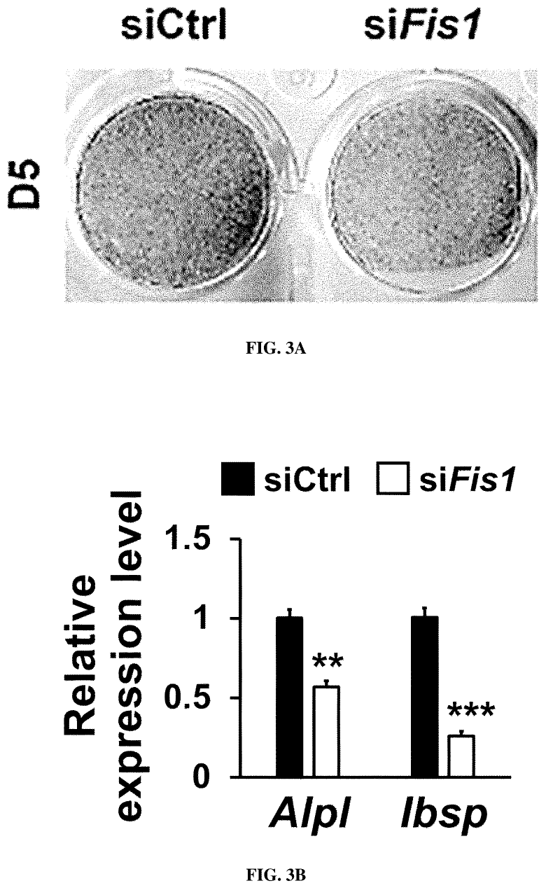

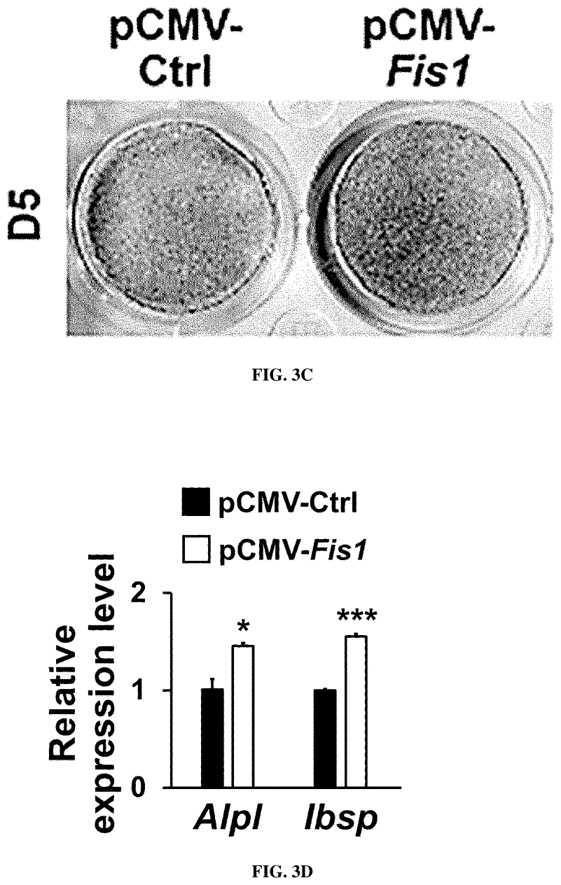

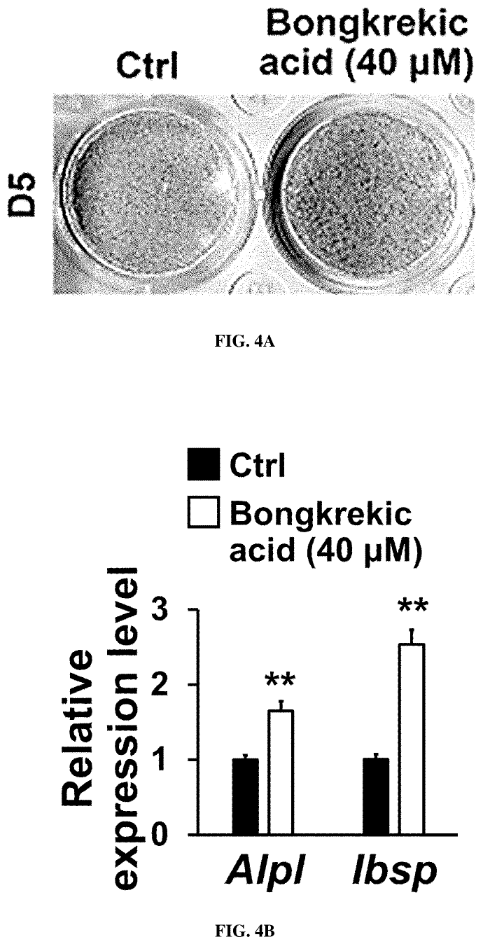

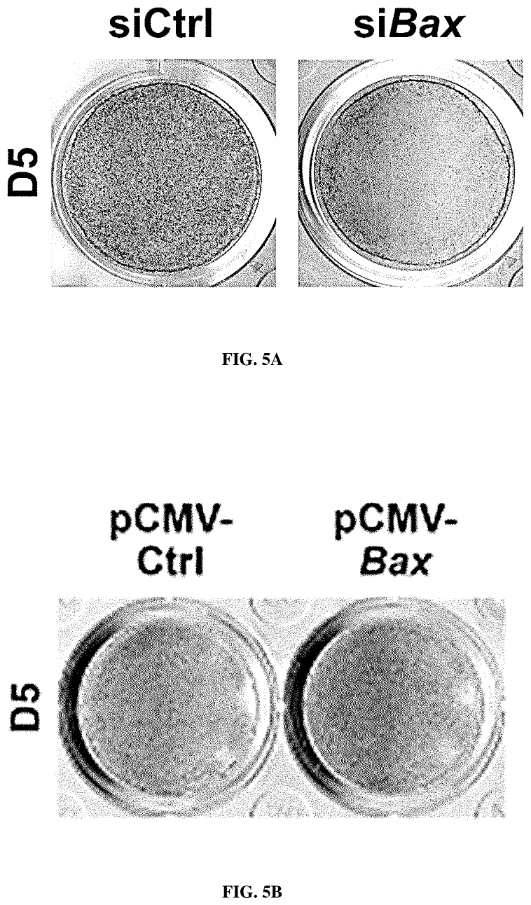

The results of observing the ALP-stained osteoblasts in Example 3-2 are shown in FIGS. 2A, 3A, 3C, 4A, 5A, and 5B. In the case of osteoblasts transfected with siOpa1, osteoblasts transfected with pCMV-Fis1 plasmid, osteoblasts transfected with pCMV-Bax plasmid, and osteoblasts treated with bongkrekic acid, the degree of staining was stronger compared to the respective controls, and in the case of osteoblasts transfected with siFis1 and osteoblasts transfected with siBax, the degree of staining was weaker compared to the respective controls.

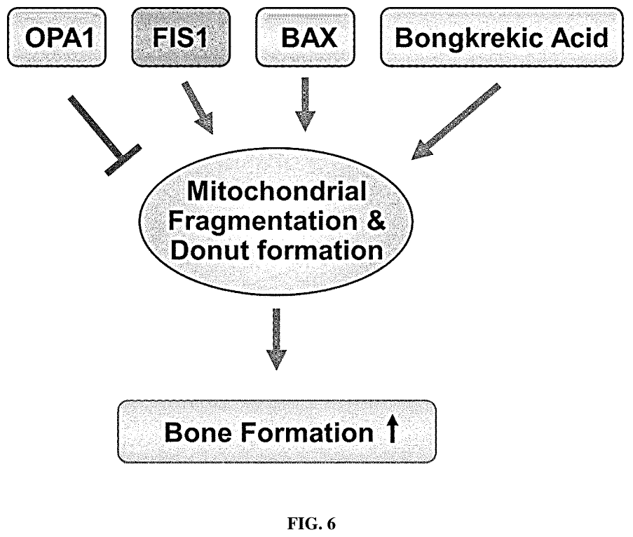

The results of evaluating gene expression levels by performing qRT-PCR in Example 3-3 are shown in FIGS. 2B, 3B, 3D, and 4B. In the case of osteoblasts transfected with siOpa1, osteoblasts transfected with pCMV-Fis1 plasmid, and osteoblasts treated with bongkrekic acid, the expression of Alp1 and Ibsp, known as osteogenic markers, was significantly increased compared to the respective controls, and in osteoblasts transfected with siFis1, the expression of Alp1 and Ibsp was significantly decreased compared to the control. Through these results, it was confirmed that inhibition of Opa1 gene expression, promotion of Fis1 gene expression, promotion of Bax gene expression, and bongkrekic acid treatment led to mitochondrial fission and formation of a donut shape, thereby increasing osteoblast bone formation activity. A mimetic diagram illustrating the effects of the genes and bongkrekic acid identified through the above experiments on mitochondria and the resulting bone formation is shown in FIG. 6.

1. A pharmaceutical composition for promoting bone formation, comprising:
an agent promoting activation of osteoblasts as an active ingredient,
wherein the agent promoting activation of osteoblasts comprises at least one selected from
1) bongkrekic acid or a pharmaceutically acceptable salt thereof,
2) an inhibitor of Opa1 or a gene encoding the same,
3) an activator of Fis1 or a gene encoding the same, and
4) an activator of Bax or a gene encoding the same.

2. The pharmaceutical composition for promoting bone formation of claim 1, wherein the inhibitor of Opa1 or a gene encoding the same is at least one selected from small-interfering RNAs (siRNAs), microRNAs (miRNAs), antisense oligonucleotides (ASOs), and aptamers targeting the Opa1 gene.

3. The pharmaceutical composition for promoting bone formation of claim 1,
wherein the activator of Fis1 or a gene encoding the same is at least one selected from mRNA encoding the Fis1 gene and a recombinant vector carrying the same, and
the activator of Bax or a gene encoding the same is at least one selected from mRNA encoding the Bax gene and a recombinant vector carrying the same.

4. The pharmaceutical composition for promoting bone formation of claim 1, wherein the pharmaceutical composition increases the proportion of osteoblast mitochondria in which the circularity value ((4×area of mitochondria)/(π×(maximum ferret diameter)²)) is 0.5 to 1 or one or more pores are formed.

5. The pharmaceutical composition for promoting bone formation of claim 1, comprising the bongkrekic acid at a concentration of 0.1 to 100 μM.

6. The pharmaceutical composition for promoting bone formation of claim 1, having an effect of increasing the expression of at least one selected from Alp1 and Ibsp.

7. A pharmaceutical composition for preventing or treating bone diseases, comprising:
an agent promoting activation of osteoblasts as an active ingredient,
wherein the agent promoting activation of osteoblasts comprises at least one selected from
1) bongkrekic acid or a pharmaceutically acceptable salt thereof,
2) an inhibitor of Opa1 or a gene encoding the same,
3) an activator of Fis1 or a gene encoding the same, and
4) an activator of Bax or a gene encoding the same.

8. The pharmaceutical composition for preventing or treating bone diseases of claim 7, wherein the inhibitor of Opa1 or a gene encoding the same is at least one selected from small-interfering RNAs (siRNAs), microRNAs (miRNAs), antisense oligonucleotides (ASOs), and aptamers targeting the Opa1 gene.

9. The pharmaceutical composition for preventing or treating bone diseases of claim 7,
wherein the activator of Fis1 or a gene encoding the same is at least one selected from mRNA encoding the Fis1 gene and a recombinant vector carrying the same, and
the activator of Bax or a gene encoding the same is at least one selected from mRNA encoding the Bax gene and a recombinant vector carrying the same.

10. The pharmaceutical composition for preventing or treating bone diseases of claim 7, wherein the pharmaceutical composition increases the proportion of osteoblast mitochondria in which the circularity value ((4×area of mitochondria)/(π×(maximum ferret diameter)²)) is 0.5 to 1 or one or more pores are formed.

11. The pharmaceutical composition for preventing or treating bone diseases of claim 7, comprising the bongkrekic acid at a concentration of 0.1 to 100 μM.

12. The pharmaceutical composition for preventing or treating bone diseases of claim 7, having an effect of increasing the expression of at least one selected from Alp1 and Ibsp.

13. The pharmaceutical composition for preventing or treating bone diseases of claim 7, wherein the bone diseases refer to fractures, osteoclasia, osteoporosis, osteomalacia, osteoarthritis, rheumatoid arthritis, or osteogenesis imperfecta.

14. A food composition for promoting bone formation, comprising:
an agent promoting activation of osteoblasts as an active ingredient,
wherein the agent promoting activation of osteoblasts comprises at least one selected
1) bongkrekic acid or a pharmaceutically acceptable salt thereof,
2) an inhibitor of Opa1 or a gene encoding the same,
3) an activator of Fis1 or a gene encoding the same, and
4) an activator of Bax or a gene encoding the same.

15. A food composition for preventing or ameliorating bone diseases, comprising:
an agent promoting activation of osteoblasts as an active ingredient,
wherein the agent promoting activation of osteoblasts comprises at least one selected from
1) bongkrekic acid or a pharmaceutically acceptable salt thereof,
2) an inhibitor of Opa1 or a gene encoding the same,
3) an activator of Fis1 or a gene encoding the same, and
4) an activator of Bax or a gene encoding the same.

16. The food composition for preventing or ameliorating bone diseases of claim 15, wherein the bone diseases refer to fractures, osteoclasia, osteoporosis, osteomalacia, osteoarthritis, rheumatoid arthritis, or osteogenesis imperfecta.

17. A method of promoting bone formation or preventing or treating a bone disease in a subject in need thereof, the method comprising administering to the subject the pharmaceutical composition of claim 1, wherein bone formation is promoted or the bone disease is prevented or treated in the subject.

18. The method of claim 17, wherein the subject has been diagnosed as having a bone fracture, osteoclasia, osteoporosis, osteomalacia, osteoarthritis, rheumatoid arthritis, or osteogenesis imperfecta.

19. The method of claim 17, wherein bone formation is being promoted in the subject.

20. The method of claim 17, wherein the bone disease is being treated in the subject.
